(19) United States
(12) Patent Application Publication
(10) Pub. No.: US 2007/0134275 A1
(43) Pub. Date: Jun. 14, 2007

(54) Medicaments for the immunotherapy of malignant tumors

(75) Inventors: Claudia Ulbrich, Hannover (DE); Klaus-Dieter Rockensuss, Fritzler (DE); Armin Grossman, Hannover (DE)

Correspondence Address: JACOBSON HOLMAN PLLC, 400 SEVENTH STREET N.W., SUITE 600, WASHINGTON, DC, 20004, US

(73) Assignee: LipoNova AG

(21) Appl. No.: 11/593,132

(22) Filed: Nov. 6, 2006

Related U.S. Application Data
(63) Continuation of application No. 09/926,630, filed on Feb. 26, 2002, now abandoned.

(30) Foreign Application Priority Data
Jul. 28, 2000 (EP) 00 116 362.5

(51) Int. Cl. A61K 39/00; C12N 5/08

(52) U.S. Cl. 424/277.1; 435/366

(57) ABSTRACT

The present invention relates to a composition which can be used as a medicament or for preparing a medicament for the immunotherapy of tumors of for tumor vaccination. The invention also relates to methods for preparing medicaments for the immuno-therapy of tumors of for tumor vaccination.

The present applications relate to compositions which are particularly suitable for the immunotherapy of malignant tumors, and methods for their preparation, and the use of the compositions for preparing medicaments.

Usually, the therapeutic treatment of tumors is effected by radical surgery, chemotherapy, radiotherapy or hormone therapy. These therapies have numerous undesirable side effects and are accompanied by significant loads on the patient. Moreover, in some tumor forms, almost no improvements are achieved with these therapies so that their use does not appear reasonable in view of the side effects. These forms include, in particular, malignant tumors, malignant melanomas, renal carcinomas, intestinal carcinomas and pancreatic carcinomas. Therefore, the mortality rate in, for example, renal carcinomas is 85%.

In recent years, knowledge has been increasingly gained on the complex interplay between tumors and the immune system, the interest becoming focused on strategies for treating tumors in which the immune system is stimulated. Generally, it is the object of such therapies to succeed in causing the immune system to recognize specific antigens from tumor cells which are not present in healthy cells, or only so to a lower extent. This is achieved, for example, by administering a medicament as described in Anticancer Research [(1997) No. 17, pages 2879-2882, and 3117-3120]: Tumor tissue is withdrawn from a patient and processed into an autologous tumor cell lysate, which is injected into the patient. This was done expecting that immunity against the tumor antigens is provided in the lysate. Another strategy is described in the published patent application WO-A 99/47687. It is disclosed therein that autologous antigen-presenting cells which express a special tumor determinant at their surfaces are injected into patients.

It is not only the object of tumor therapies to prevent the growth of tumors and the formation of metastases, but also to promote their regression. The patient's expectation of life is to be prolonged, and his health and life quality improved. About the success of immune therapies, it can be said at present that the therapeutic treatments used so far, unfortunately, can achieve success only in single cases or only in part. It is a basic problem that many tumor markers are also present in healthy cells in particular stages of differentiation and in certain amounts. Therefore, activation of the immune system against such tumor markers often does not occur to the extent desired or with the required specificity.

It has been the object of the present invention to develop medicaments for tumor therapy which achieve the above mentioned objects to a high extent. Also, when the medicaments according to the invention are used, it should be possible to perform tumor therapies relatively quickly and simply.

The present invention relates to a composition for the immunotherapy of tumors. The composition can be obtained by a process in which tumor material is evaluated, comminuted and transferred into a purified cell suspension, which is then incubated with interferon-gamma and tocopherol acetate and frozen to form a tumor cell lysate, and in which monocytes are isolated from buffy coats or whole blood and subsequently induced to differentiation into dendritic cells by incubation with cytokines and transferred into the non-adherent stage, whereupon a calculated amount of the above frozen tumor cell lysate is thawed, added as an antigen, cytokines are added, incubation is performed, and the mature dendritic cells produced are harvested.

“Evaluation” of the tumor material means macroscopic evaluation of the tissue, upon which clearly discernible proportions of adipose, connective and functional renal tissues, blood vessels and other non-tumor tissues are identified and subsequently removed and discarded.

In a particular embodiment, autologous tumor material is used for producing the composition. When the composition is produced, IL-4 and GM-CSF and/or IFN-gamma are preferably added to immature dendritic cells for differentiation.

The composition according to the invention is especially suitable as a medicament or for the preparation of a medicament for immunotherapy. Medicaments containing the cell lysate according to the invention are preferably injected intracutaneously or subcutaneously.

All conceivable types of solid tumor diseases can be treated with the medicament according to the invention. Medicaments containing the composition according to the invention are especially suitable for the treatment of tumors in which other treatment methods are little successful. In particular, in addition to other malignant solid tumors, these include malignant melanomas, renal carcinomas, intestinal carcinomas, pancreatic carcinomas, lymphomas, bronchial carcinomas and gynecological tumors.

When patients are treated with the medicament according to the invention within the scope of a tumor therapy, unexpectedly pronounced positive effects for the patients are observed. The growth of tumors and the formation of metastases could be prevented to a surprisingly high extent while the regression of tumors was promoted. The health, life quality and expectation of life of the patients were clearly increased. These effects can be achieved probably because the medicament according to the invention is distinct from known ones in essential aspects. One particular difference from many known methods is that tumor markers are not simply administered to the patient, but directly introduced into the patient's immune system in dendritic cells as vehicles. Surprisingly, it is sufficient to add a crude cell lysate of tumor cells to the dendritic cells in vitro, whereas according to WO-A-99/47687, antigen-presenting cells are admixed or transfected with a purified antigen. Therefore, the method according to the invention can also be performed more quickly and more simply as compared to known methods. This is especially important in the preparation of such therapeutic substances in order to keep the risk of contaminations low. In addition, the cell lysate has the advantage that the whole antigen repertoire of a tumor cell is available.

The present invention also relates to methods for the preparation of a medicament in which a suspension of tumor cells is prepared, the tumor cells are killed, and monocytes are isolated from blood, their differentiation into dendritic cells is induced, and the thus obtained “immature” dendritic cells are incubated with the cell lysate of the killed tumor cells, the maturing of the dendritic cells is induced, and the “mature” dendritic cells are harvested.

The monocytes are preferably isolated from buffy coats, from separated stem cells, from leukapheretic products, or from whole blood.

The differentiation of the monocytes into “immature” dendritic cells is preferably induced by cytokines, IL-4 and GM-CSF. Especially suitable for induction of the maturing from “immature” to “mature” dendritic cells are prostaglandin E₂ and TNF-α and/or IL-1β and IL-6 in addition to IL-4 and GM-CSF. The preparation of the tumor cell suspensions is generally effected by isolating and optionally evaluating tumor material, which is then comminuted and transferred into a purified cell suspension. In a particular embodiment of the method according to the invention, the suspension of tumor cells is prepared from autologous tumor material. In another preferred embodiment, the expression of membrane-borne protein complexes is induced in the tumor cell suspension prior to said killing of the tumor cells. The induction is preferably effected by interferon-gamma and tocopherol acetate. The killing of the tumor cells is effected, in particular, by freezing. The harvesting of the mature dendritic cells is preferably performed when typical morphological characteristics are present (e.g., veil formation) as evaluated by microscopic check and/or by characterization of surface antigens using fluorescent antibodies. The invention also relates to the use of the described composition and its possible embodiments for preparing medicaments for tumor therapy. According to the invention, the composition described and its possible embodiments are also used for the preparation of medicaments for tumor vaccination.

### EXAMPLE

### Preparation of a Composition for Tumor Therapy

A) Preparation of a Tumor Cell Lysate

For preparing the tumor tissue, proportions of adipose, connective and functional renal tissues as well as blood vessels and necrotic tissues which are clearly discernible macroscopically are carefully removed and discarded. The ready prepared tissue is comminuted to a size as small as possible (pieces of about 2-3 mm diameter) and/or enucleated and then transferred into a sterile sieve (50-100 mesh) together with the surrounding medium. With a glass rod, a tissue pieces present in the sieve are passed through with slow stirring without pressure. The passed cells are transferred into a sterile beaker with medium RPMI 1640, and after addition of 15 ml of RPMI medium (RPMI 1640 with 25 mmol HEPES) into the sieve, the tissue remnants in the sieve are again passed through with a glass rod.

The cell suspension is layered onto 45% Percoll cushion. This step serves for the removal of any erythrocytes present and for the enrichment of mononuclear cells on the Percoll cushion. The filled tubes are centrifuged, and the interphase with the mononuclear cells is sucked off, transferred into a tube, pelletized by centrifugation and washed with NaCl/glucose solution. The total number of vital cells is determined microscopically using a Neubauer counting chamber after staining of the cells with trypan blue. In addition, cell typing is performed using TestSimplets® (Boehringer Mannheim), which are suitable for rendering carcinoma cells distinguishable from other cells in a quick staining process. After resuspension of the cells in sodium chloride/glucose solution, vitamin E (700 μg/dose to be prepared) and interferon-gamma (1500 IU/dose to be prepared) are added. The mixture is incubated in a water bath at 37° C. for two hours, centrifuged and washed twice with sodium chloride/glucose solution. The mixture is aliquoted into cryotubes and converted to a tumor cell lysate by freezing at −85° C.±5° C. The quality controls comprise the tests according to specification for cell count, sterility and devitalization.

B) Preparation of the Dendritic Cells and of the Composition for Tumor Therapy

Media employed:
Medium A: RPMI medium+1% autologous plasma
Medium B: medium A+GM-CSF (800 U/ml)+IL-4 (1000 U/ml)
Medium C: medium B+TNF-α(1000 U/ml)+prostaglandin E₂ (1 μg/ml).

The buffy coats from released blood donations, from leukaphereses or whole blood from a blood bag are transferred into centrifuge tubes and centrifuged. The interphase contains the mononuclear cells (=buffy coat) and is separated from the erythrocytes (bottom) and the plasma (top). The plasma and mononuclear cells are layered on Lymphoprep® (Nycomed) and centrifuged. Subsequently, the plasma and mononuclear cells are pipetted off and again centrifuged. The plasma is taken off and used for preparing the media. Residual plasma is stored at from +2° C. to +8° C. in order to prepare additional medium A, if needed. The cell pellet is washed twice with NaCl solution (0.9%) and centrifuged. Prior to the second washing step, vital cells are counted after staining with trypan blue. The centrifugation residue is taken up in medium A at a cell concentration of 4×10⁶/ml. The cell suspension is applied to Petri dishes and incubated at 37° C.±1° C. and 5% CO₂ for two hours. A microscopic check for adherent cells (monocytes) is then effected, whereupon medium A is carefully sucked off to remove non-adherent cells.

Medium B is added to the Petri dishes, followed by incubation at 37° C.±1° C. and 5% CO₂. On day 1, medium B is sucked off, and fresh medium is added. On day 2, medium B is sucked off partially (3 ml), and fresh medium B (3 ml) is added. On day 5, a microscopic check is effected to see whether adherent cells have undergone transition to the non-adherent stage. The cells of one charge are combined, and vital cells are counted after staining with trypan blue. The cells are centrifuged off and taken up in a calculated amount (5×10⁵/well/3 ml) in medium C, the volume corresponding to one tenth of the final volume, a calculated amount of tumor cell lysate (5×10⁴/well/3 ml) is added, followed by homogenization and incubation for one hour at 37° C.±1° C. and 5% CO₂, and then medium C is filled to the final volume. The cell suspension is plated on 6-well plates and further incubated at 37° C./5% CO₂. On days 6 and 7, a microscopic check is performed: the maturing process of the dendritic cells starts to show by “veil formation”. On day 8, the mature dendritic cells are “harvested” upon microscopic check when the “veil formation” has become pronounced. The mature dendritic cells are pelletized by centrifugation and washed twice. The centrifugation residue is taken up in 0.9% NaCl solution, vital cells are counted after staining with trypan blue, and 0.9% NaCl solution is used to adjust the desired cell count.

1-16. (canceled)

17. A composition obtainable by a process
in which renal-system-tumor material is evaluated, comminuted and made into a purified cell suspension, which is then incubated with interferon-gamma and tocopherol acetate and frozen to form a tumor cell lysate and
in which monocytes are isolated from buffy coats or whole blood and subsequently induced to differentiate into dendritic cells by incubation with cytokines and converted to the non-adherent stage,
whereupon the tumor cell lysate is thawed and thawed lysate, containing fragments of the tumor cells, and cytokines are added to the non-adherent dendritic cells, followed by incubation of the admixture and harvesting of the mature dendritic cells.

18. The composition according to claim 17, wherein IL-4 and GM-CSF are added for differentiation into inmature dendritic cells in the preparation.

19. A medicament containing the composition according to claim 17 in combination with an acceptable carrier.

20. A method for preparing a medicament in which a suspension of renal-system-tumor cells is prepared, which is then incubated with interferon-gamma and tocopherol acetate, the tumor cells are killed, and monocytes are isolated from blood, their differentiation into dendritic cells is induced, and the thus obtained immature dendritic cells are incubated with the cell lysate of the killed tumor cells, the maturing of the dendritic cells is induced, and the mature dendritic cells are harvested as the medicament.

21. The method according to claim 20, in which the monocytes are isolated from buffy coats, whole blood, leukaphereses, or separated stem cells.

22. The method according to claim 20, in which the differentiation of the monocytes into immature dendritic cells is induced by cytokines, IL-4 and GM-CSF with or without interferon-gamma.

23. The method according to claim 20, in which the maturing from immature to mature dendritic cells is induced by prostaglandin E₂ and TNF-α and/or IL-1β and IL-6 in addition to IL-4 and GM-CSF.

24. The method according to claim 20, in which the tumor cell suspension is prepared by isolating renal-system-tumor material, which is then comminuted and made into a purified cell suspension.

25. The method according to claim 20, in which the tumor cells are killed by freezing.

26. The method according to claim 20, in which the mature dendritic cells are harvested when typical morphological characteristics of cell maturity are present as evaluated by microscopic check and/or by characterization of surface antigens using fluorescent antibodies.

27. A method of using the composition according to claim 17 for tumor therapy comprising administering the composition to a patient.

28. The method of claim 27, wherein the renal-system-tumor material is autologous.

29. The method according to claim 20, in which the tumor cell suspension is prepared by isolating and evaluating renal-system-tumor material, which is then comminuted and made into a purified cell suspension.
